(19) United States
(12) Patent Application Publication
(10) Pub. No.: US 2006/0079892 A1
(43) Pub. Date: Apr. 13, 2006

(54) Adjustable tandem connectors for corrective devices for the spinal column and other bones and joints

(76) Inventors: Suranjan Roychowdhury, Plymouth, MN (US); Duy Nguyen, Savage, MN (US); Jeffrey Cota, St. Louis Park, MN (US)

Correspondence Address: FAEGRE & BENSON;ATTN: PATENT DOCKETING, 2200 WELLS FARGO CENTER, 90 SOUTH 7TH STREET, MINNEAPOLIS, MN, 55402-3901, US

(21) Appl. No.: 10/238,877

(22) Filed: Sep. 10, 2002

Related U.S. Application Data
(60) Provisional application No. 60/334,771, filed on Oct. 31, 2001.

(51) Int. Cl. A61F 2/30

(52) U.S. Cl. 606/61

(57) ABSTRACT

Connectors for interconnecting rods, fixed to vertebrae and other bones of a subject include a body portion and two recesses or pockets for receiving the rods. These connectors are generally able to be secured to the rods by a tightening a fastener or retaining member from above the spine, which can facilitate the procedure for the surgeon. The connectors can include rotatable or stationary pockets, mating members that pivot and/or translate within the coronal plane of the subject to adjust to the positions of the rods, and extension shafts that pivot to adjust for rod angle.

### CROSS-REFERENCE TO PROVISIONAL APPLICATION

This application claims the benefit of Provisional Application Ser. No. 60/334,771, filed Oct. 31, 2001 entitled Adjustable tandem connectors for corrective devices for the spinal column and other bones and joints, the disclosure of which is hereby incorporated herein by reference in its entirety as if set forth fully herein.

### FIELD OF THE INVENTION

The present invention relates generally to devices for correcting the spinal column and other bones and joints, and more specifically to connectors for such devices.

### BACKGROUND OF THE INVENTION

The bones and connective tissue of an adult human spinal column consist of an upper portion (the cervical, thoracic, and lumbar regions) having more than 20 discrete bones, and a lower portion which consists of the sacral bone and the coccygeal bodies. The bones of the upper portion are generally similar in shape, the size of the bones progressively varying from small to large downwardly along the spine.

The vertebrae are coupled to one another by a tri-joint complex consisting of an anterior disc and the two posterior facet joints, the anterior discs of adjacent bones being cushioned by cartilage spacers referred to as intervertebral discs. Referring now to FIGS. 1, 2 and 3 (which are reproductions of FIGS. 1-3 of U.S. Pat. No. 5,885,284, top, lateral, and posterior views, respectively, of typical vertebral bones of the spinal column are shown. The spinal cord is housed in the central canal 10, protected from the posterior side by a shell of bone called the lamina 12. The lamina 12 has three large protrusions. Two of these extend laterally from the side ends thereof and are referred to as the transverse processes 14. The third extends back and down from the center of the lamina and is called the spinous process 16. The lamina 12 defines an arched shape about the posterior of the spinal cord, the arched shape having lateral portions 13a, 13b which are generally straight, and which meet beneath the spinous process 16 at a curved surface 15.

The anterior portion of the spine comprises a set of generally cylindrically shaped bones which are stacked one on top of the other. These portions of the vertebrae are referred to as the vertebral bodies 20, and are each separated from the other by the intervertebral discs 22. Pedicles 24 are bone bridges which couple the anterior vertebral body 20 to the corresponding lamina 12 and transverse and spinous processes 14, 16.

Referring specifically to FIG. 3, the stacking of vertebrae is shown from the posterior. As can be seen in FIG. 3, each vertebra is coupled to the one above and below via facet joints 19 on either side of an opening into the spinal canal 10.

In its entirety, the spinal column is highly complex in that it houses and protects critical elements of the nervous system which have innumerable peripheral nerves and arterial and venous bodies in close proximity. In spite of these complexities, the spine is a highly flexible structure, capable of a high degree of curvature and rotation through a wide range of motion. Genetic or developmental irregularities, trauma, chronic stress, tumors, and disease, however, can result in spinal pathologies which either limit this range of motion or threaten the critical elements of the nervous system housed within the spinal column.

Such pathologies may be treated by a wide variety of therapeutic interventions, including immobilization of one or more vertebrae. A variety of systems have been proposed which achieve this immobilization by implanting artificial assemblies in, or on, the spinal column. These assemblies may be classified by their position relative to the spine as anterior, posterior, or lateral implants. Anterior and lateral assemblies generally comprise short structures which support only a few adjacent vertebral bodies. Conversely, posterior implants often comprise pairs of elongate vertically disposed rods for stabilizing both short and long segments of the spine. Typically, such posterior rods are coupled to the back of the spinal column via hooks which slip under the lamina, means for attachment to the transverse process, and/or by screws which are inserted through the pedicle (often termed “pedicle screws”).

In some instances it may be desirable to provide enhanced torsional rigidity to the rods. In such instances, cross-linking devices or connectors which couple the rods together transverse to the axes of the rods are typically employed. Exemplary devices and connectors are illustrated in U.S. Pat. No. 5,885,284 to Errico et al., U.S. Pat. No. 5,084,049 to Asher et al., U.S. Pat. No. 5,752,955 to Errico et al., U.S. Pat. No. 6,136,003 to Hoeck et al., U.S. Pat. No. 6,113,600 to Drummond et al., and U.S. Pat. No. 5,368,594 to Martin et al. In other instances, such as revision or extension procedures, it may be desirable to utilize a connector to connect new hardwired to the existing rods or plates. Exemplary devices and connectors commercially available from DePuy Acromed (e.g., Moss Miami axial connectors and Isola Aval rod connectors) and Sofamor Danek (e.g., CROSSLINK® multi-span plates and offset plates, TSRH offset plates, and CD Horizon axial/domino connectors). In considering the design of a connector, issues include its size (because the connector is implanted in the body near the spine, it should occupy a relatively small volume in order that it be comfortable and non-intrusive for the patent) and its ease of implantation (which can encompass both the ease of attaching an individual rod to the connector and its orientation relative to the patient and the doctor during surgery). Because (a) there are a number of different surgical procedures in which these connectors are employed and (b) different surgeons have different preferences for connector configurations, it is desirable to provide new connector configurations that can meet individual surgeons' needs.

### SUMMARY OF THE INVENTION

Connector embodiments of the present invention are configured to address different surgical needs and techniques for interconnecting multiple bone fixation devices (such as rods or plates between vertebrae). As a first aspect, the present invention is directed to a connector that comprises: first and second mating members, each of the members including a body portion, a mating projection and a recess adapted to engage a respective one of at least two bone fixation rods; first and second retaining members; and a fastener. The body portions of the mating members include an aperture having a longitudinal axis that is generally perpendicular to longitudinal axes of the rods, and the mating projections of the first and second mating members include an aperture, the mating projection of the first mating member overlying the mating projection of the second mating member such that their respective apertures are generally axially aligned. The first and second retaining members are inserted into, respectively, the body portion apertures of the first and second mating members to engage a respective rod. The fastener is inserted through the mating projection apertures of the first and second mating members. When the fastener is in a tightened condition, the first and second mating members are prevented from relative rotation, and when the fastener is in a loosened condition, the first and second mating members are free to rotate about an axis of rotation that is generally parallel to the longitudinal axes of the body portion apertures of the first and second mating members. In this configuration, the connector can be manipulated in the coronal plane of the subject to interconnect non-parallel rods, and the rods can be secured and the connector tightened easily by a surgeon from above the spine.

As a second aspect, a connector of the present invention that can interconnect at least two bone fixation rods comprises: a body portion, a mating projection and a recess adapted to engage a first bone fixation rod; an extension shaft; and a fastener. The mating projection of the body portion has an aperture having a longitudinal axis generally perpendicular to the longitudinal axis of the first rod. The extension shaft has a shank with a slot therein, the shank being inserted into and rotatable relative to the mating portion aperture, and the slot being adapted to receive a second bone fixation rod. The fastener engages the shank of the extension shaft. When the fastener is in a tightened condition, the fastener, shank and mating projection engage the second rod and prevent relative movement thereof, and preferably, when the fastener is in a loosened condition, the second bone fixation rod is free to slide relative to the mating projection parallel to the longitudinal axis of the rod. A retaining member (such as a set screw) can be used to secure the first rod in the recess. This connector embodiment also enables a surgeon to secure bone fixation rods from above the spine.

As a third aspect, a connector of the present invention for interconnecting bone fixation rods comprises: first and second mating members; first and second retaining components; and a fastener. Each of the first and second mating members includes a body portion, a mating projection and a rod pocket adapted to engage a respective rod. Each of the mating projections of the first and second mating members includes an aperture, and the mating projection of the first mating member overlies the mating projection of the second mating member such that their respective apertures are generally axially aligned. The rod pockets of the first and second mating members having openings positioned above the body portion and facing in a first direction that is generally perpendicular to an axis located between the first and second rod pockets. The first and second retaining components (such as set screws) couple from above with, respectively, the rod pockets of the first and second mating members to secure a bone fixation respective rod. The fastener is inserted through the mating projection apertures of the first and second mating members. This configuration also enables a surgeon to secure bone fixation rods from above while engaging bone fixation rods from below.

As a fourth aspect, a connector embodiment of the present invention for interconnecting bone fixation rods comprises: a body portion; first and second rod pockets; and first and second retaining components. Each of the rod pockets includes an opening positioned above the body portion and facing in a first direction generally perpendicular to the rod longitudinal axes. The first rod pocket is rotatably attached with one end of the body portion such that the first rod pocket is rotatable relative to the body portion over at least two axes of rotation. The first and second retaining components couple from above with, respectively, the rod pockets of the first and second mating members to secure a respective rod. This connector configuration enables the surgeon to adjust the position and angle of the rod pocket to meet the positional requirements of the rods, and to do so from beneath the rods, while retaining the ability to tighten and secure the rods from above the spine.

As a fifth aspect, a connector embodiment of the present invention for interconnecting bone fixation rods comprises: a body portion; a cover portion; and a clamping component. The body portion has a pair of recesses, each recess being configured to engage a respective rod, and further comprises a post (preferably threaded). The cover portion overlies the body portion and has a pair of recesses, each recess being configured to align with a respective body portion recess and engage a respective rod, as well as an aperture that receives the body portion post. The clamping component has an aperture (again, preferably threaded) that receives the body portion post. The clamping component is movable to a tightened condition in which the clamping component applies pressure to the cover portion, thereby clamping the rods between the cover portion and the body portion.

### BRIEF DESCRIPTION OF THE FIGURES

FIG. 1 is a top view of a vertebra of the human spine.

FIG. 2 is a side view of a series of vertebrae of the human spine.

FIG. 3 is a posterior view of a series of vertebrae of the human spine.

FIG. 4 is a perspective view of a connector embodiment of the present invention joining two bone fixation rods.

FIG. 5 is an exploded perspective view of the connector embodiment of FIG. 4.

FIG. 6 is a perspective view of another connector embodiment of the present invention joining two bone fixation rods.

FIG. 7 is an exploded perspective view of the connector embodiment of FIG. 6.

FIG. 8 is a perspective view of an additional connector embodiment of the present invention joining two bone fixation rods.

FIG. 9 is an exploded perspective view of the connector embodiment of FIG. 8.

FIG. 10 is a perspective view of a further connector embodiment of the present invention joining two bone fixation rods.

FIG. 11 is an exploded perspective view of the connector embodiment of FIG. 10.

FIG. 12 is a perspective view of still another connector embodiment of the present invention joining two bone fixation rods.

FIG. 13 is an exploded perspective view of the connector embodiment of FIG. 12.

FIG. 14 is a perspective view of yet another connector embodiment of the present invention joining two bone fixation rods.

FIG. 15 is an exploded perspective' view of the connector embodiment of FIG. 14.

FIG. 16 is a perspective view of another connector embodiment of the present invention joining two bone fixation rods.

FIG. 17 is an exploded perspective view of the connector embodiment of FIG. 16.

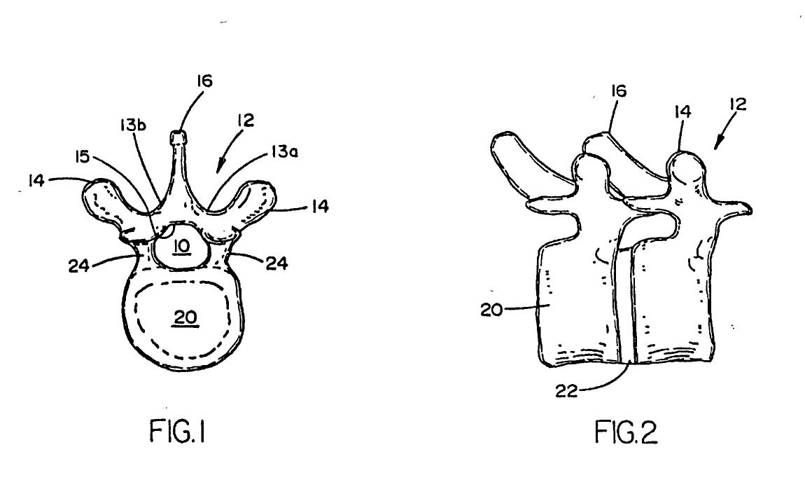

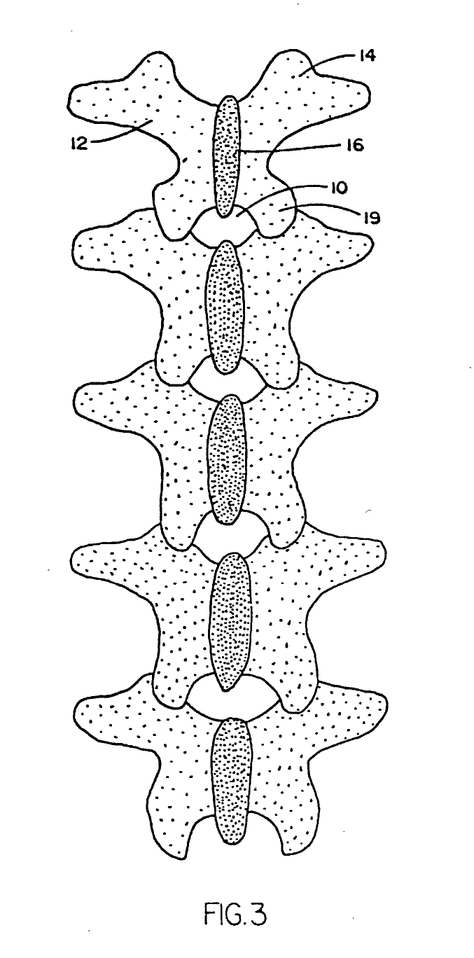

### DETAILED DESCRIPTION OF THE INVENTION

The present invention will now be described more fully hereinafter, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art. In the drawings, like numbers refer to like elements throughout. Thicknesses and dimensions of some components may be exaggerated for clarity.

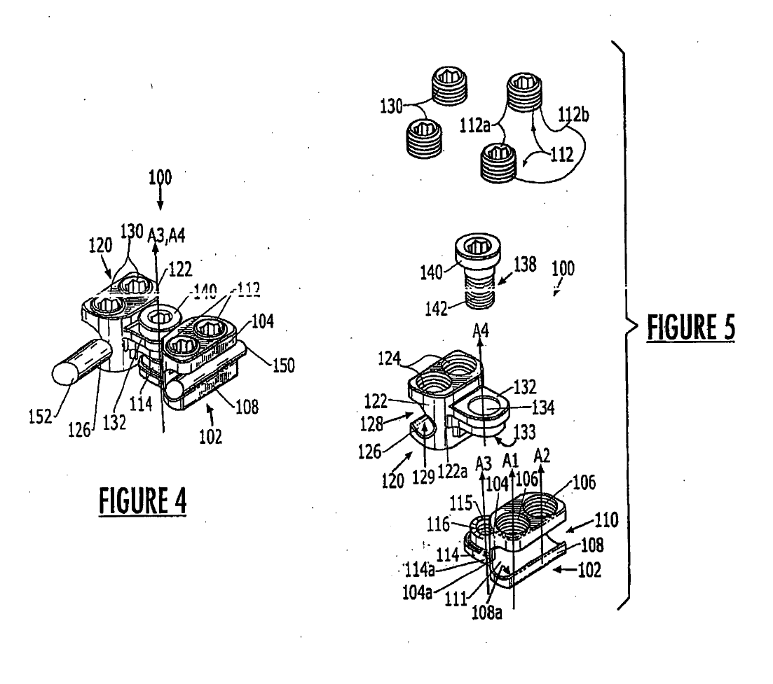

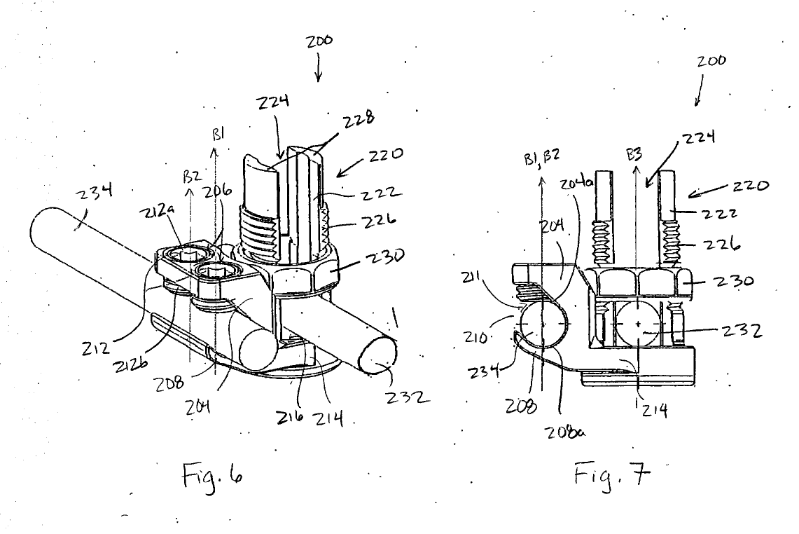

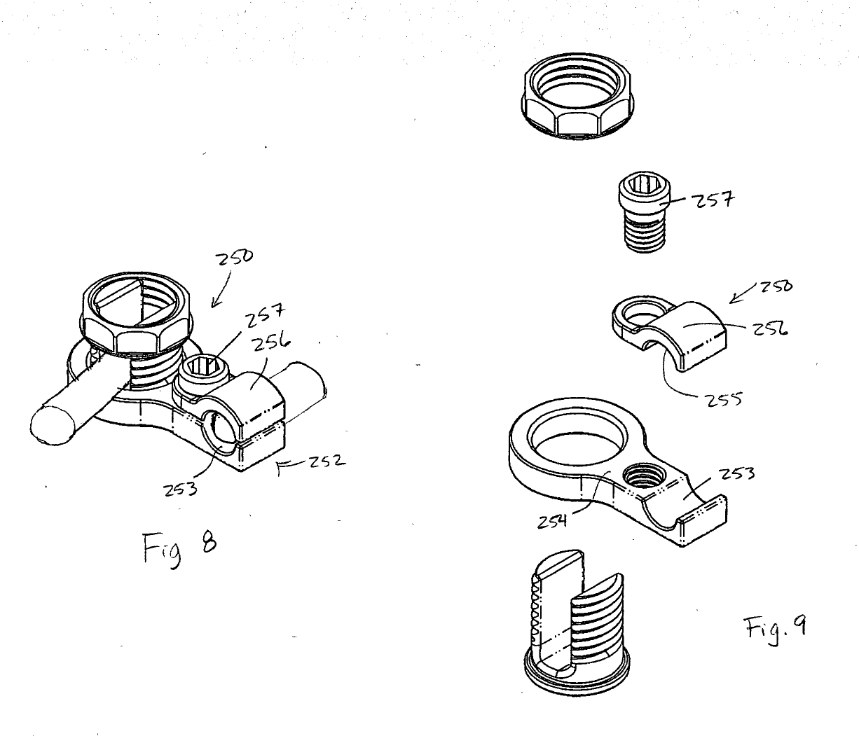

Turning now to the figures, a connector, designated broadly at 100, is illustrated in FIGS. 4 and 5. The connector 100 includes a first mating member 102, a second mating member 120, and a bolt 138. These components are described in detail below.

The first mating member 102 includes a body portion 104, a finger 108, and a mating projection 114. The body portion 104 is generally wedge-shaped and includes a pair of apertures 106, each of which has a respective longitudinal axis A1, A2. The finger 108 extends from one end of the body portion 104. The free end of the finger 108 and the lower edge of the body portion 104 form an opening 110 that leads to a recess 111 defined by an arcuate upper surface 108a of the finger 108 and a substantially flat lower surface 104a of the body portion 104. Two set screws 112 extend through the apertures 106; the heads 112a of the set screws 112 extend above the body portion 104 when in a loosened condition, and the shanks 112b of the set screws 112 extend into the recess 111. The mating projection 114 extends away from a lower region 114a of the body portion 104 in a direction generally opposite that of the finger 108. The mating projection 114 has serrations 115 on its upper surface. A threaded aperture 116 extends through the mating projection 114; the aperture 116 includes a longitudinal axis A3 that is generally parallel with the axes A1, A2. The mating projection 114 includes a cutaway portion 118.

The second mating member 120 is the mirror image of the first mating member 102 with the exception of the location and configuration of its mating projection. More specifically, the second mating member 120 includes a body portion 122 with threaded apertures 124, a finger 126 that, with the body portion 122, forms an opening 128 into a recess 129, and set screws 130 that extend through the apertures 124. The mating projection 132 of the second mating member 120 extends from an upper region 122a of the body portion 122 in a direction generally opposite that of the finger 126. The mating projection 132 includes serrations 133 on its lower surface that are configured to mate with the serrations 115 of the mating projection 114 of the first mating member 102. A non-threaded aperture 134 extends through the mating projection 132 and has a longitudinal axis A4 that is substantially coincident with the axis A3. When the second mating member 120 is attached to the first mating member 102, the mating projection 132 overlies the mating projection 114; also, the mating projection 132 is complimentary to and nests within the recess 109 of the first mating member 102, and the mating projection 114 is complimentary to and nests within the recess 135 of the second mating member 120.

The bolt 138 has a head 140 and a threaded shank 142. The shank 142 extends through the aperture 134 and is threadedly received in the aperture 116, such that the head 140 resides above the mating projection 132.

As can be seen in FIG. 4, the connector 100 can be attached to two rods 150, 152 that have been mounted to vertebrae of a subject. In most instances, the connector 100 will be oriented such that the head 140 of the bolt 138 faces away from the spine. After the attachment of the rods 150, 152 to respective vertebrae, the connector 100 is positioned so that one of the rods 150 is engaged within the recess 111, and the other rod 152 is engaged within the recess 129. In each instance, the rods 150, 152 can be inserted into their respective recesses 111, 129 through the openings 110, 128 located on the sides of the first and second mating members 102, 120. The bolt 138 should be in a loosened condition, thereby enabling the first and second mating members 102, 120 to rotate relative to each other about the axes A3, A4 (ie., the first and second mating members 102, 120 are free to rotate relative to each other within the coronal plane of the subject) to accommodate non-parallel rods 150, 152.

After the rods 150, 152 are positioned within their respective recesses 111, 129, they can be secured therein through the tightening of the set screws 112, 130. Notably, the set screws 112, 130 are oriented so that the tightening heads thereof face the same direction (ie., away from the spine) as the head 140 of the bolt 138. This orientation typically will cause the heads of the set screws 112, 130 to face the surgeon, thereby facilitating tightening of the set screws 112, 130.

After the set screws 112, 130 are tightened, the bolt 138 can then be tightened into the aperture 116 to force the mating projections 114, 132 together. The compression of the mating projections 112, 130 causes the serrations 115, 133 to nest and mate, thereby preventing relative rotation of the first and second members 102, 120. Like that of the set screws 112, 130, the head 140 of the bolt 138 faces the surgeon to facilitate tightening.

Of course, the connector 100 can be attached to the rods 150, 152 in a different sequence of steps than that described above. For example, one or both of the rods can be attached to the connector 100 prior to attachment of the rod to a vertebra of the subject. Also, the bolt 138 may be tightened prior to the tightening of the set screws 112, 130, or the bolt 138 and/or the set screws 112, 130 may be tightened to less than full torque during insertion, then tightened to a higher torque magnitude after all components have been inserted. The skilled artisan will understand that other sequences of steps for insertion may also be suitable.

Notably, the connector 100 is configured such that the centers of the recesses (i.e., the locations where the centers of the rods 150, 152 reside within the recesses 111, 129) are located at substantially the same depth relative to the top surfaces of the body portions 104, 122. This results from the offset relationship of the mating projections 114, 130, in which the mating projection 114 extends from the lower region of the body portion 104, while the mating projection 130 extends from the upper region of the body portion 122. Because the centers of the rods 150, 152 are located at approximately the same depth in the subject, the connector 100 can be recessed farther from the subject's dorsal skin surface than some prior connectors that lack this offset design.

Each of the mating members 102, 120 is typically formed as a unitary component, preferably of titanium, titanium alloys (like Ti-6Al-7Nb), nickel titanium alloys, cobalt chromium alloys, or other suitable metallic materials. In most instances, the rods 150, 152 will be located on the same side of the subject, i.e., they will be located on the same side of a plane defined by the spinous processes of the vertebrae of the subject. As such, the dimensions of the connector 100 should remain relatively small; typically, the distance between the centers of the recesses 111, 129 is typically between about 0.35 inch and about 1.2 inches. This distance can reduced somewhat by the nesting of the mating projections 114, 132 within complimentary recesses in the body portions 104, 122. The depth of the recesses 111, 129 (from the front of the recess to its rear) is preferably between about 0.3 and 1 inch.

Those skilled in this art will recognize that alternative embodiments of the connector that differ from that illustrated herein may also be suitable. For example, the serrations 115, 133 may be replaced with roughened surfaces, knurls, or other nesting topography that prevents relative rotative movement. The pairs of set screws 112 may be replaced with a single set screw, bolts or other retaining members. The bolt 138 may be replaced with a screw, a bolt/nut combination, or another fastener.

Turning now to FIGS. 6 and 7, another connector embodiment, designated broadly at 200, is illustrated therein. The connector 200 includes a body portion 204, a finger 208, and a mating projection 214. The body portion 204 is generally wedge-shaped and includes a pair of apertures 206, each of which has a respective longitudinal axis B1, B2. The finger 208 extends from one end of the body portion 204. The free end of the finger 208 and the lower edge of the body portion 204 form an opening 210 that leads to a recess 211 defined by an arcuate lower surface 208a of the finger 208 and a substantially flat upper surface 204a of the body portion 204. Two set screws 212 extend through the apertures 206; the heads 212a of the set screws extend above the body portion 204 when in a loosened condition, and the shanks 212b of the set screws 212 extend into the recess 211.

The mating projection 214 extends away from a lower region of the body portion 204 in a direction generally opposite of that of the finger 208. The mating projection 214 includes a threaded aperture 216 having a longitudinal axis B3 that is generally parallel with the axes B1, B2.

An extension shaft 220 includes a shank 222 having a slot 224 and external threads 226. The extension shaft 220 is configured such that the threaded end of the shank 220 is threaded into the aperture 216 of the connector 200. The illustrated extension shaft 220 is of the “break-off” variety, which includes tabs 228 that can be snapped off after insertion of a nut 230 to save space within the subject.

As illustrated in FIGS. 6 and 7, the connector 200 and the extension shaft 220 can be used to interconnect two rods 232, 234. After the rods 232, 234 are mounted in the subject, the extension shaft 220 is threaded into the aperture 216 (within which it is freely rotatable) until the slot 224 takes an orientation that enables the rod 234 to reside therein. The rod 232 is inserted into the recess 211 through the side opening 210 and secured therein in the manner described above for the connector 100. The nut 230 is then threaded onto the external threads 226 of the extension shaft 220 and tightened to secure the rod 234 within the slot 224 of the shank 222. Thus, the connector 200 can interconnect two rods to provide additional stability to the spine and can be adjusted within the coronal plane. As with the connector 100, other sequences of steps for insertion of the connector 200 and rods 232, 234 may also be suitable.

Like the connector 100, the connector 200 can be relatively simple for the surgeon to insert and secure, as the set screws 212 and the nut 230 all face away from the spine and can be tightened conveniently by the surgeon. It also is notable that the connector 200 enables the centers of the rods 232, 234 to be located at essentially the same depth in the subject, which, as described above, may be desirable.

The connector 200 is preferably formed as a unitary component, preferably of titanium, titanium alloys (like Ti-6Al-7Nb), nickel titanium alloys, cobalt chromium alloys, or other suitable metallic materials. The dimensions are typically such that the rod centers are separated by between about 0.35 inch and about 1.2 inches and the depth of the recesses is between about 0.3 and 1 inch.

Alternative embodiments of the connector 200 include those employing screws or bolts instead of the set screws and those having jam nuts or set screws in place of the nut 230. Of course, in these alternative embodiments, the extension shaft may have interior threads or other projections/recesses to mate with the fastener of choice. In addition, an alternative embodiment illustrated in FIGS. 8 and 9 and designated broadly at 250 includes a two piece “claw” 252 formed by a recess 253 in the body portion 254 and a recess 255 in a cover portion 256. The claw 252 is held together by a set screw 257. The remaining structure of the connector 250 matches that of the connector 200, and the discussion above directed to the connector 200 and its alternative embodiments is equally applicable to the connector 250.

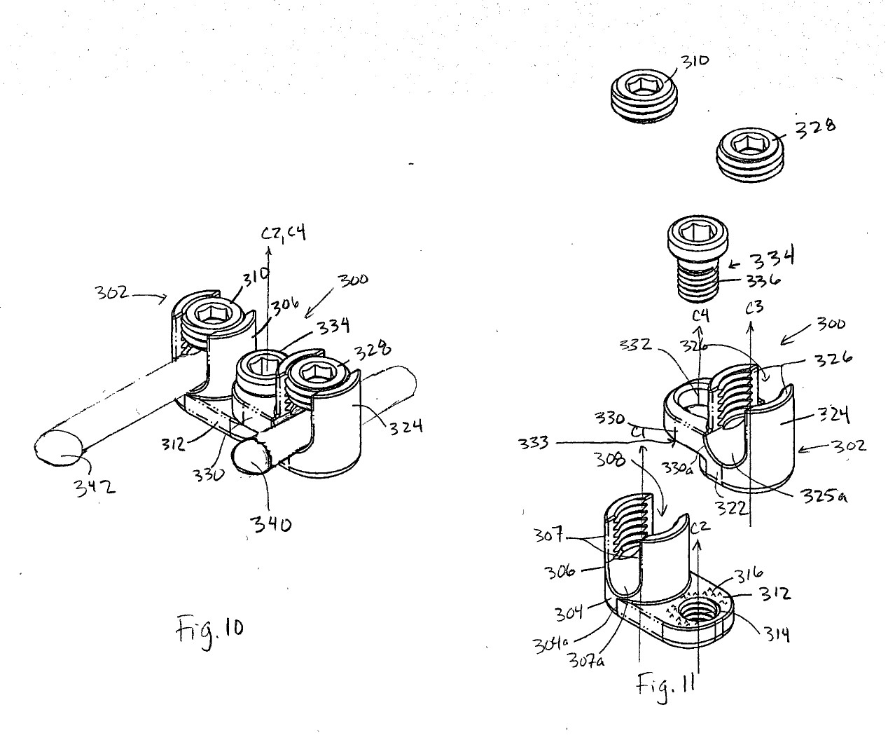

Another connector embodiment, designated broadly at 300, is illustrated in FIGS. 10 and 11. The connector 300 includes first and second mating members 302, 320 and a bolt 334. These components are further described below.

The first mating member 302 includes a body portion 304 to which is attached a pocket 306. The pocket 306 is defined by side walls 307 and a floor 307a, and has an opening 308 opposite the floor 307a. An axis C1 extends from the floor 307a through the opening 308. A set screw 310 fits within the opening 308 to close the pocket 306. A mating projection 312 extends from a lower region 304a of the body portion 304 in a direction away from the pocket 306. The mating projection 312 has a threaded aperture 314 with a longitudinal axis C2. The mating projection 312 also has knurls 316 on its upper surface.

The second mating member 320 includes a body portion 322 and a pocket 324 formed by side walls 325 and a floor 325a. An opening 326 is defined by the side walls 325 opposite the floor 325a; an axis C3 extends from the floor 325a through the opening 326. A set screw 328 is positioned in the opening 326 to close the pocket 324. A mating projection 330 originates at an upper region 330a of the body portion 322 and extends away from the pocket 324. The mating projection 330 has an elongate aperture 332 that overlies the aperture 314; the aperture 332 has a longitudinal axis C4 that is generally parallel with the longitudinal axis C2. The mating projection 330 also has knurls 333 on its lower surface that, when the mating projection 330 overlies the mating projection 312, mate with the knurls 316 of the mating projection 312.

The bolt 334 is inserted through the aperture 332 and is threadedly received in the aperture 314. The bolt 334 is sized such that its shank 336 is smaller in diameter than the width of the aperture 332. In a loosened condition, the first and second mating members 302, 320 are free to rotate about the axes C1, C2 and to translate laterally relative to one another (i.e. such that the distance between the pockets 306, 324 can be increased or reduced). Typically, the distance between the centers of the pockets 306, 324 will be range between about 0.35 and about 1.2 inches.

In use, the connector 300 is inserted into the subject such that rods 340, 342 are secured within the pockets 306, 324. This is accomplished by positioning the connector 300 below the rods 340, 342 (ie., such that the pockets 306, 324 face away from the spine and toward the surgeon) without the set screws 310, 328 in place. The bolt 334 should be in a loosened condition. The connector 300 is then raised and the first and second mating members 302, 320 rotated and translated relative to each other so that the rods 340, 342 are inserted into the pockets 306, 324. The bolt 334 is then tightened to prevent movement of the first and second mating members 302, 320 relative to each other; this movement is further prevented by the interaction of the knurls 315, 333 on the mating projections 312, 330 of the first and second mating members 302, 320. The set screws 310, 328 are placed in the openings 308, 326 and tightened to secure the rods 340, 342 in place. Those skilled in this art will recognize that other sequences of steps for inserting the connector 300 and rods 340, 342 may also be suitable.

Those skilled in this art will recognize that alternative configurations of the connector 300 may also be suitable for use. For example, the set screws maybe replaced with jam nuts or nuts threaded onto the outer surfaces of the side walls 307, 325 of the pockets 306, 324. The bolt 334 may be replaced by a screw or other threaded fastener. The elongate aperture 332 of the mating projection 330 of the second mating member 320 may be non-elongate if relative translation of the first and second mating members 302, 320 is not required or desired. Also, alternative surface topography (such as roughened mating surfaces) may be used on the mating projections 312, 330 in place of the knurls 315, 333.

Each of the mating members 302, 320 is typically formed as a unitary component, preferably of titanium, titanium alloys (like Ti-6Al-7Nb), nickel titanium alloys, cobalt chromium alloys, or other suitable metallic materials. In many instances, the rods 340, 342 will be located on the same side of the subject, i.e., they will be located on the same side of a plane defined by the spinous processes of the vertebrae of the subject. As such, the dimensions of the connector 300 should remain relatively small; typically, the distance between the centers of the pockets 306, 324 is typically between about 0.35 and about 1.2 inches.

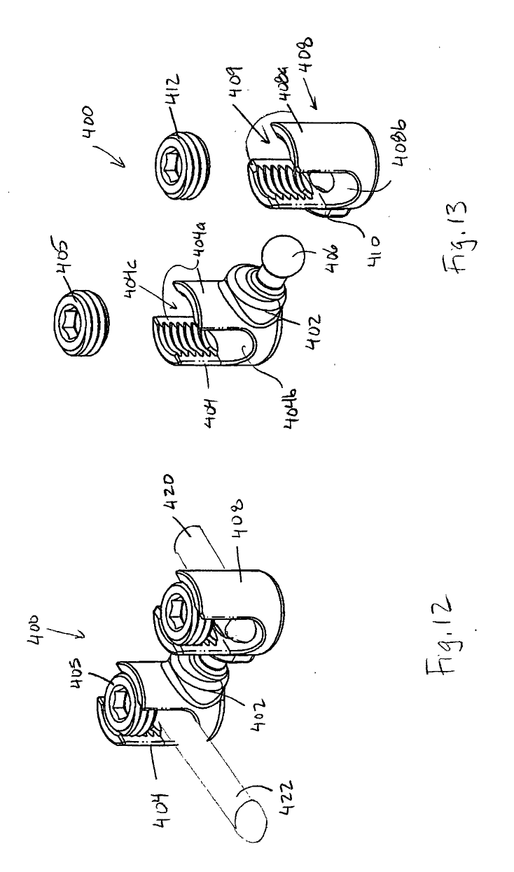

A further connector embodiment, designated broadly at 400, is illustrated in FIGS. 12 and 13. The connector 400 includes a body portion 402, a stationary pocket 404, and a rotatable pocket 408. The stationary pocket 404, which is located at one end of the body portion 402, is defined by side walls 404a and a floor 404b; the side walls 404a define an opening 404c that is opposite the floor 404b. A set screw 405 is received within and covers the opening 404c.

The body portion 402 includes a ball 406 at the end thereof opposite the stationary pocket 404. The rotatable pocket 408 is rotatably attached to the ball 406 through a socket 410 that enables the pocket 408 to rotate relative to the body portion 402 about multiple axes of rotation. The rotatable pocket 408 also includes side walls 408a and a floor 408b; the side walls 408a define an opening 409 opposite the floor 408b. A set screw 412 is received within and covers the opening 409.

In operation, the connector 400 is disposed in the subject in the same manner as the connector 300; i.e., it is positioned such that the body portion 402 resides nearer the spine of the subject than the openings 404c, 409 of the pockets 404, 408. With the set screws 405, 412 removed from the openings 404c, 409, rods 420, 422 can be inserted within the pockets 404, 408, with the rotatable pocket 408 being capable of rotating to different orientations relative to the stationary pocket 404 to adjust for the location and/or angle of the rod 422 relative to the rod 420. Once the rods 420, 422 are inserted into their respective pockets 404, 408, they can be secured by the insertion and tightening of the set screws 405, 412 in the openings 404c, 409; tightening of the set screw 412 also should prevent further rotation of the rotatable pocket 408 relative to the body portion 402. The orientation of the connector 400 can should cause the set screws 405, 412 to face the surgeon, thereby facilitating their tightening and, consequently, the securing of the connector 400 to the rods 420, 422. As with the other connector embodiments discussed above, the sequence of steps for inserting the connector 400 and rods 420, 422 in the subject may be varied.

Those skilled in this art will recognize that alternative configurations of the connector 400 may also be suitable for use. For example, the set screws may be replaced with jam nuts or nuts threaded onto the outer surfaces of the side walls 404a, 408a of the pockets 404, 408. Also, the ball 406 may take a different configuration, such as one that enables the rotatable pocket to be “locked” into preferred orientations.

Typically, the body portion 402 (with the ball 406) and the stationary pocket 404 are formed as one unitary component, while the rotatable pocket 408 is formed as a separate component. Both of these components are preferably formed of titanium, titanium alloys (like Ti-6Al-7Nb), nickel titanium alloys, cobalt chromium alloys, or other suitable metallic materials. The dimensions of the connector 400 will typically be such that the distance between the centers of the pockets 404, 408 is between about 0.35 and about 1.2 inches, with the rotatable pocket 408 being able to pivot over a range of about 0 to about 270 degrees in the coronal plane, about 0 to about 360 degrees in the sagittal plane, and about 0 to about 360 degrees in the transverse plane.

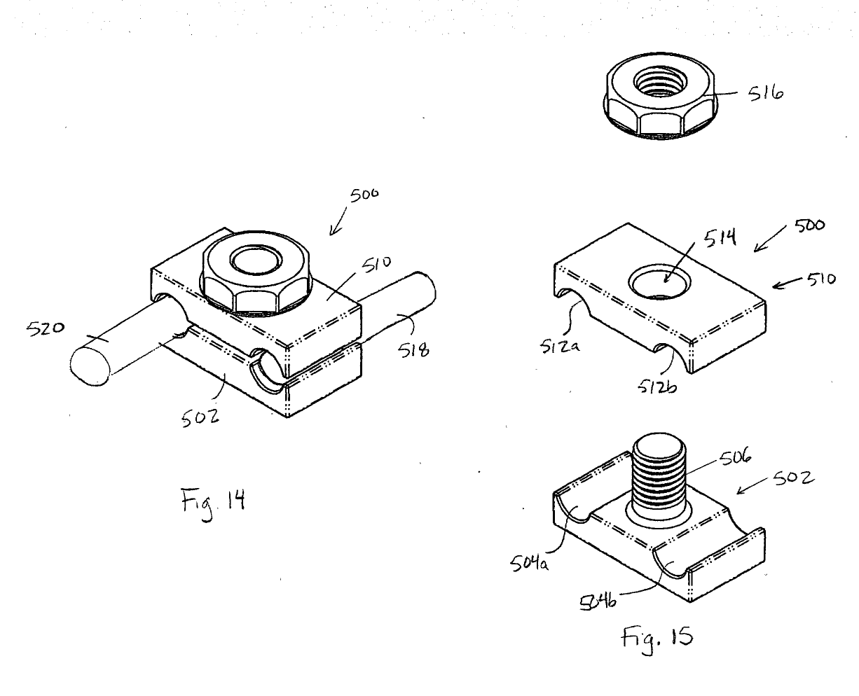

Still another connector embodiment of the present invention, designated broadly at 500, is illustrated in FIGS. 14 and 15. The connector 500, which is a non-adjustable connector (ie., with respect to rotation and lateral translation), includes a body portion 502, a cover portion 510, and a nut 516. The body portion 502 has a pair of upwardly-facing recesses 504a, 504b located on opposite sides thereof. An externally threaded post 506 rises from the center of the body portion 502. The cover portion 510 includes a pair of downwardly-facing recesses 512a, 512b and a centrally-located aperture 514. The nut 516 is internally threaded and sized to receive the post 506.

In use within a subject, the body portion 502 is positioned to underlie two rods 518, 520 attached to the spine of a patient, with the rods 518, 520 being positioned in respective recesses 504a, 504b. The cover portion 510 is then positioned to overlie the body portion 502 so that the post 506 extends through the aperture 514 and the recesses 512a, 512b overlie the rods 518, 520. The nut 516 is then threaded onto the post 506 to secure the rods 518, 520 within the connector 500.

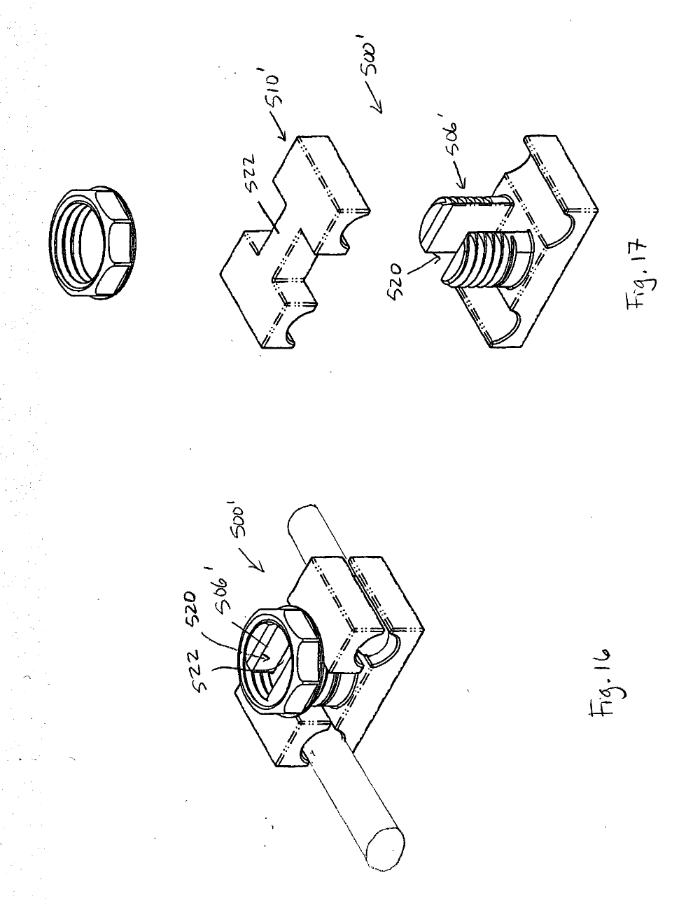

As an alternative configuration to that of the post 506 and aperture 514, as shown in FIGS. 16 and 17 in a connector 500′, the post 506′ can include a slot 520, and the cover portion 510′ can include a bridge 522 that is received within the slot 520.

The body portion 502 and the cover portion 510 are typically formed as unitary components and preferably of titanium, titanium alloys (like Ti-6Al-7Nb), nickel titanium alloys, cobalt chromium alloys, or other suitable metallic materials. The distance between the centers of the rods 518, 520 is preferably between about 0.35 and about 1.2 inches.

Those skilled in this art will appreciate that, although the connectors of the present invention are illustrated herein for use in spinal correction, they may also be used to correct or supplement other orthopedic procedures. Also, although such procedures are typically performed on human subjects, veterinary surgeries also may benefit from the use of these connectors.

The foregoing is illustrative of the present invention and is not to be construed as limiting thereof. Although exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention.

1. An apparatus for connecting vertebrae of a subject, comprising:
at least two rods, each of the rods defining a longitudinal axis; and
a connector that interconnects the rods, the connector comprising:
first and second mating members, each of the members including a body portion, a mating projection, and a recess adapted to engage a respective one of the rods, the body portion including an aperture having a longitudinal axis that is generally perpendicular to the longitudinal axes of the rods, each of the mating projections of the first and second mating members including a mating surface having a nesting topography, an outer surface opposing the mating surface, and an aperture, wherein the aperture of the second mating member is a threaded aperture and the mating projection of the first mating member is superposed onto the mating projection of the second mating member such that the mating surfaces of the mating projections and their respective apertures are generally axially aligned;
first and second retaining members inserted into, respectively, the body portion apertures of the first and second mating members to engage a respective rod; and
a fastener having a widened portion on the first end and a threaded portion on the second end inserted through the mating projection apertures of the first and second mating members, the widened portion of the fastener in engagement with the outer surface of the mating projection of the fist mating member and the threaded portion in engagement with the threaded aperture in the mating projection of the second mating member;
wherein, when the fastener is in a tightened condition and the nesting topographies of the superposed mating surfaces engage each other, the first and second mating members are prevented from relative rotation, and when the fastener is in a loosened condition, the first and second mating members are free to rotate about an axis of rotation that is generally parallel to the longitudinal axes of the body portion apertures of the first and second mating members.

2. The apparatus defined in claim 1, wherein the nesting topography of the mating surfaces includes a plurality of serrations that inhibit relative rotation of the first and second mating members when the fastener is in the tightened condition.

3. The apparatus defined in claim 1, wherein the mating projection apertures are configured such that, when the fastener is in the loosened condition, the first and second mating members are free to translate relative to each other along a translation axis that is perpendicular to the axis of rotation.

4. The apparatus defined in claim 1, wherein the first and second retaining members are threaded fasteners accessible from a first side of the connector, and wherein the fastener is accessible from the first side of the connector.

5. The apparatus defined in claim 1, wherein each of the first and second mating members further comprises a finger, the fingers and the body portions of the first and second mating members together forming, respectively, the recesses of the first and second mating members.

6. The apparatus defined in claim 1, wherein each of the body portions has an upper surface, and the mating projections of the first and second mating members are located at different first and second distances from their respective body portion upper surfaces.

7. The apparatus defined in claim 6, wherein each of the body portion recesses has a center, and wherein the recess centers are positioned at substantially the same distance from the body portion upper surfaces.

8. (canceled)

9. The apparatus defined in claim 1, wherein each of the body portions of the first and second mating members has a cutaway portion that is complementary to and nested with an end of the mating projection of the other of the first and second mating members.

10. A method of attaching support rods to the vertebrae of a subject, comprising:
attaching first and second support rods to separate vertebrae of the subject, the first and second support rods being generally parallel and attached to the same side of a plane formed by a spinous process of the subject's vertebrae;
providing a connector, the connector comprising:
first and second mating members, each of the members including a body portion, a mating projection and a recess that is adapted to engage a respective one of the first and second support rods, the body portion including an aperture having a longitudinal axis that is generally perpendicular to the longitudinal axes of the rods, each of the mating projections of the first and second mating members including a mating surface having a nesting topography, an outer surface opposing the mating surface, and an aperture, the mating projection of the first mating member superposed onto the mating projection of the second mating member such that the mating surfaces of the mating projections and their respective apertures are generally axially aligned;
first and second retaining members inserted into, respectively, the body portion apertures of the first and second mating members to engage a respective first or second support rod; and
a fastener having a widened portion on a first end and a threaded portion on a second end inserted through the mating projection apertures of the first and second mating members, the widened portion of the fastener engaging the outer surface of the mating projection of the first mating member and the threaded portion engaging the threaded aperture in the mating projection of the second mating member;
wherein, when the fastener is in a tightened condition, the nesting topographies of the mating surfaces engage each other, and the first and second mating members are prevented from relative rotation, and when the fastener is in a loosened condition, the first and second mating members are free to rotate about an axis of rotation that is generally parallel to the longitudinal axes of the apertures of the body portions of the first and second mating members; and
engaging the first support rod with the recess of the first mating member;
rotating the second mating member relative to the first mating member to facilitate engagement of the second mating member with the second support rod;
engaging the second support rod with the recess of the second mating member, and
tightening the fastener to its tightened condition to prevent relative rotation of the first and second mating members.

11. An apparatus for interconnecting vertebrae of a subject, comprising:
first and second rods, each of the rods defining a longitudinal axis; and
a connector, the connector comprising:
a body portion, a mating projection and a recess adapted to engage the first rod, the mating projection having an aperture having a longitudinal axis generally perpendicular to the longitudinal axes of the rods;
an extension shaft having a shank with a slot therein, the shank being inserted into and rotatable relative to the mating portion aperture, the slot receiving the second rod; and
a fastener that engages the shank of the extension shaft;
wherein when the fastener is in a tightened condition, the fastener, shank and mating projection engage the second rod and prevent relative movement thereof.

12. The apparatus defined in claim 11, wherein when the fastener is in a loosened condition, the second rod is free to slide relative to the mating projection parallel to the longitudinal axis of the rod

13. The apparatus defined in claim 11, wherein the body portion includes an aperture with a longitudinal axis generally perpendicular to the longitudinal axes of the rods, and further comprising a retaining member that extends through the aperture in the body portion and contacts the first rod, thereby retaining the first rod in engagement within the recess.

14. The apparatus defined in claim 11, wherein the extension shaft shank is threaded.

15. The apparatus defined in claim 11, wherein the recess is configured such that the first rod may be inserted in the recess after the fastener is tightened to its tightened condition.

16. An apparatus for connecting vertebrae of a subject, comprising:
at least two rods, each of the rods defining a longitudinal axis; and a connector that interconnects the rods, the connector comprising: first and second mating members, each of the members including a body portion, a mating projection and a rod pocket adapted to engage a respective one of the rods, each of the mating projections of the first and second mating members including an aperture, the mating projection of the first mating member overlying the mating projection of the second mating member such that their respective apertures are generally axially aligned, the rod pockets of the first and second mating members having openings positioned above the body portion and facing in a first direction that is generally perpendicular to an axis located between the first and second rod pockets;
first and second retaining components that couple from above with, respectively, the rod pockets of the first and second mating members to engage a respective rod; and
a fastener inserted through the mating projection apertures of the first and second mating members.

17. The apparatus defined in claim 16, wherein, when the fastener is in a tightened condition, the first and second mating members are prevented from relative rotation, and when the fastener is in a loosened condition, the first and second mating members are free to rotate about an axis of rotation that is generally parallel to the first direction.

18. The apparatus defined in claim 16, wherein the first and second retaining members are set screws.

19. An apparatus for connecting vertebrae of a subject, comprising:
at least two rods, each of the rods defining a longitudinal axis; and
a connector that interconnects the rods, the connector comprising:
a body portion and first and second rod pockets, each of the rod pockets including an opening positioned above the body portion and facing in a first direction generally perpendicular to the rod longitudinal axes, the first rod pocket being rotatably attached with one end of the body portion such that the first rod pocket is rotatable relative to the body portion over at least two axes of rotation; and
first and second retaining components that couple from above with, respectively, the rod pockets of the first and second mating members to engage a respective rod.

20. The apparatus defined in claim 18, wherein the body portion includes a partially spherical surface and the first rod pocket includes a complimentary partially spherical surface, the mating of the partially spherical surfaces enabling the first rod pocket to rotate relative to the body portion over a limited range about multiple axes of rotation.

21. An apparatus for connecting vertebrae of a subject, comprising: at least two rods, each of the rods defining a longitudinal axis; and
a connector that interconnects the rods, the connector comprising:
a body portion having a pair of recesses, each recess configured to engage a respective rod, the body portion further comprising a post;
a cover portion overlying the body portion and having a pair of recesses, each recess configured to align with a respective body portion recess and engage a respective rod, the cover portion further comprising an aperture that receives the body portion post; and
a clamping component having an aperture that receives the body portion post, the clamping portion being movable to a tightened condition in which the clamping component applies pressure to the cover portion, thereby clamping the rods between the cover portion and the body portion.

22. The apparatus defined in claim 20, wherein the body portion post is threaded, and the clamping component is a nut threadedly received on the nut.

23. A connector for interconnecting bone fixation rods, comprising:
first and second mating members, each of the members including a body portion, a mating projection and a recess adapted to engage a respective one of at least two bone fixation rods, the body portion including an aperture having a longitudinal axis that is generally perpendicular to longitudinal axes of the rods, each of the mating projections of the first and second mating members including an aperture, the mating projection of the first mating member overlying the mating projection of the second mating member such that their respective apertures are generally axially aligned;
first and second retaining members inserted into, respectively, the body portion apertures of the first and second mating members to engage a respective rod; and
a fastener inserted through the mating projection apertures of the first and second mating members;
wherein, when the fastener is in a tightened condition, the first and second mating members are prevented from relative rotation, and when the fastener is in a loosened condition, the first and second mating members are free to rotate about an axis of rotation that is generally parallel to the longitudinal axes of the body portion apertures of the first and second mating members.

24. A connector for interconnecting bone fixation rods, the connector comprising:
first and second members, each including a retainer portion adapted to engage a respective one of at least two bone fixation rods and a projection having a first surface with a nesting topography and a second surface opposing the first surface, the first and second members forming a pivotal linkage wherein the projections are positioned adjacent to each other along their nesting topographies, and
a fastener operatively connected to the linkage, wherein, when the fastener is in a tightened condition and the nesting topographies engage each other, the second surfaces of each of the first and second members are free from engagement with the other member, and the first and second members are prevented from relative pivotal motion, and when the fastener is in a loosened condition, the first and second members are free to move relative to each other.

25. The method of claim 10, wherein the step of engaging the second support rod comprises positioning the second support rod on same side of a plane defined by the spinous processes of the vertebrae of the subject as the first support rod.
